SEQUENCE LISTING (2 sequences)

SEQ ID NO: 1 - RNA, 1608 nt, Homo sapiens
  source 1..1608: mol_type = mRNA
atggcgacctgcatttggctgcggagctgtggggcccggcgcctcgggtcgacgtttccaggctgccgcctccgcccccgcgccggcggcctggtccctgcctccgggcctgcgcccggcccggcccagctccgctgctacgctgggcgcctggcgggcctctctgcggcgctgctgcgcaccgacagcttcgtgggcggccgctggctcccggccgccgccaccttccccgtgcaagacccggccagcggcgccgctctgggcatggtagccgactgcggggtgcgagaggcccgcgccgccgtgcgcgctgcctacgaggctttctgccgctggagggaggtctccgccaaggagaggagttcattacttcggaagtggtacaatttaatgatacaaaataaggatgaccttgccagaataatcacagctgaaagtggaaagccactgaaggaggcacatggagaaattctctattccgcctttttcctagagtggttctctgaggaagcccgccgtgtttacggagacattatccacaccccggcaaaggacaggcgggccctggtcctcaagcagcccataggcgtggctgcagtcatcaccccgtggaatttccccagtgccatgatcacccggaaggtgggggccgccctggcagccggctgtactgtcgtggtgaagcctgccgaagacacgcccttctccgccctggccctggctgagcttgcaagccaggctgggattccttcaggtgtatacaatgttattccctgttctcgaaagaatgccaaggaagtaggggaggcaatttgtactgatcctctggtgtccaaaatttcctttactggttcaacaactacaggaaagatcctgttgcaccacgcagcaaactctgtgaaaagggtctctatggagctgggcggccttgctccatttatagtatttgacagtgccaacgtggaccaggctgtagcaggggccatggcatctaaatttaggaacactggacagacttgtgtttgctcaaaccaattcttggtgcaaaggggcatccatgatgcctttgtaaaagcattcgccgaggccatgaagaagaacctgcgcgtaggtaatggatttgaggaaggaactactcagggcccattaattaatgaaaaagcggtagaaaaggtggagaaacaggtgaatgatgccgtttctaaaggtgccaccgttgtgacaggtggaaaacgacaccaacttggaaaaaatttctttgagcctaccctgctgtgcaatgtcacccaggacatgctgtgcactcatgaagagactttcgggcctctggcaccagttatcaagttcgatacagaggaggaggctatagcaatcgctaacgcagctgatgttgggttagcaggttatttttactctcaagacccagcccagatctggagagtggcagagcagctggaagtgggcatggttggcgtcaacgaaggattaatttcctctgtggagtgcccttttggtggagtgaagcagtccggccttgggcgagaggggtccaagtatggcattgatgagtatctggaactcaagtatgtgtgttacgggggcttgtag

SEQ ID NO: 2 - protein, 535 aa, Homo sapiens
MATCIWLRSCGARRLGSTFPGCRLRPRAGGLVPASGPAPGPAQLRCYAGRLAGLSAALLRTDSFVGGRWLPAAATFPVQDPASGAALGMVADCGVREARAAVRAAYEAFCRWREVSAKERSSLLRKWYNLMIQNKDDLARIITAESGKPLKEAHGEILYSAFFLEWFSEEARRVYGDIIHTPAKDRRALVLKQPIGVAAVITPWNFPSAMITRKVGAALAAGCTVVVKPAEDTPFSALALAELASQAGIPSGVYNVIPCSRKNAKEVGEAICTDPLVSKISFTGSTTTGKILLHHAANSVKRVSMELGGLAPFIVFDSANVDQAVAGAMASKFRNTGQTCVCSNQFLVQRGIHDAFVKAFAEAMKKNLRVGNGFEEGTTQGPLINEKAVEKVEKQVNDAVSKGATVVTGGKRHQLGKNFFEPTLLCNVTQDMLCTHEETFGPLAPVIKFDTEEEAIAIANAADVGLAGYFYSQDPAQIWRVAEQLEVGMVGVNEGLISSVECPFGGVKQSGLGREGSKYGIDEYLELKYVCYGGL

(19) United States
(12) Patent Application Publication
(10) Pub. No.: US 2025/0064900 A1
(43) Pub. Date: Feb. 27, 2025

(54) MESSENGER RNA TREATMENT FOR SUCCINIC SEMIALDEHYDE DEHYDROGENASE DEFICIENCY

(71) Applicant: Washington State University, Pullman, WA (US)

(72) Inventors: Jean-Baptiste Roullet, Pullam, WA (US); Pranav Nawani, Pullman, WA (US)

(21) Appl. No.: 18/809,940

(22) Filed: Aug. 20, 2024

Related U.S. Application Data
(60) Provisional application No. 63/520,769, filed on Aug. 21, 2023.

(51) Int. Cl. A61K 38/44; C12N 9/02

(52) U.S. Cl. CPC A61K 38/44; C12N 9/0008; C12Y 102/01024

(57) ABSTRACT

Provided herein is an mRNA therapy composition and method for use thereof to treat succinic semialdehyde dehydrogenase (SSADH) deficiency (SSADHD). ALDH5A1-coding mRNA is formulated and encapsulated in lipid nanoparticles (LNPs) or micelles, then administered to subjects with SSADHD to effect expression of ALDH5A1, restoring SSADH activity and resulting in neurometabolic improvement.

### TECHNICAL FIELD

This disclosure relates to succinic semialdehyde dehydrogenase (SSADH) deficiency (SSADHD) and messenger ribonucleic acid (mRNA) therapy for the treatment thereof.

### BACKGROUND

SSADHD is the most prevalent inherited disorder of γ-aminobutyric acid (GABA) metabolism. Few patients (˜300) have been reported worldwide, but due to underreporting the true prevalence is likely higher considering the nonspecific neurological phenotype and potential for misdiagnosis. SSADHD typically presents with infantile onset hypotonia and developmental delay, and later severe expressive language impairment, intellectual deficiency, ataxia, seizures, sleep disturbances, and neuropsychiatric problems predominantly manifest by marked anxiety and obsessive-compulsive symptoms. SUDEP (sudden unexplained death of epilepsy) has been reported in 10-15% of adolescents and adults. The neurometabolic consequences of SSADH deficiency (increased GABA and GHB, decreased GABA receptor expression) are key determinants of clinical severity and disease burden, and disease burden is extreme both for patients and caregivers. It follows that developing treatments with curative potential are warranted to address considerable and urgent medical needs.

Target pathway and pathogenesis. SSADHD is caused by homozygous or compound heterozygous mutations in the ALDH5A1 gene (610045) on chromosome 6p22 resulting in impaired degradation of succinic semialdehyde and accumulation of both GABA and γ-hydroxybutyric acid (GHB) (see FIG. 1). The biochemical hallmark of SSADHD is an elevation of blood and tissue concentrations of GABA, GHB and GABA-derived metabolites. Brain PET imaging studies with [¹¹C]-flumazenil further showed decreased GABA_{A} receptor (GABA_{A}R) binding, suggesting down-regulation of GABA_{A}Rs secondary to elevated brain GABA levels. Evidence of decreased expression of GABA_{A}R subunits in aldh5a1−/− mice and in autopsied patient brain further supports a compensatory decrease in GABA_{A}R-mediated signaling. Concentrations of the excitatory neurotransmitter glutamate in the brain are variable but there is strong evidence of pronounced deficiency in brain glutamine, the metabolic precursor of glutamate and an essential component of the astrocyte-neuron GABA/glutamate recycling pathway. Causation of brain glutamine depletion in SSADHD is unknown, but studies conducted in KO mice show that a glutamine-rich diet has no impact on disease phenotype and no impact on brain glutamine. Studies also show that glutamine supplementation has no impact on brain astrogliosis (hypertrophicd astrocytes), a hallmark of mouse and human SSADHD. Low brain glutamine levels may be caused by disease-induced astrocyte dysfunction. Astrocytes maintain the proper balance between excitatory glutamate and inhibitory GABA and buffer excess ammonia by controlling the glutamate-ammonia-glutamine-GABA cycle. Other studies show that inhibition of glutamine synthesis is a protective mechanism against astrocyte swelling and cellular hypertrophy. Regardless of the cause of astrogliosis in SSADHD, it is a response to injury as seen in other brain disorders such as stroke. Hence, a decrease in brain astrogliosis is a marker of treatment efficacy.

Current treatments. There is no cure for SSADHD, and treatments are exclusively symptomatic. Pharmacologic treatment is generally aimed at ameliorating symptoms of the disease, primarily seizures and psychiatric sequalae. Currently employed symptomatic interventions primarily target seizures, behavioral symptoms (ADHD, OCD, anxiety), sleep and GI disorders, but patients are also taking numerous dietary supplements, the efficacy of which in treating disease-related symptoms remains unconfirmed. The anti-epileptic vigabatrin is sometimes used but it is a last-recourse drug for refractory seizures because of a significant risk for ocular toxicity. The use of valproic acid, another antiepileptic drug, is also limited because of the potential for inhibition of residual SSADH activity.

Several investigational targeted therapeutics have been considered, e.g. NCS-382, bumetanide, ganaxolone, farnesol, taurine, torin-2, and SGS-742, but the clinical potential of these compounds remains unconfirmed or inconclusive. Enzyme replacement therapy (ERT) and gene therapy have yet to achieve clinical success, despite studies in KO mice treated with recombinant SSADH which show improvement in survival and decrease in brain GHB levels. Despite promising findings, ERT has yet to demonstrate its potential in controlled clinical trials and would only correct circulating enzyme levels. Successful adenoviral (AV)-mediated gene therapy has been reported in the KO mouse model with increased survival rates, but because of its known limitations and transient nature, AV-mediated therapy has not moved to the clinical realm. Further, gene therapy does not allow gene dose titration with repeated administrations at different maturational ages.

There is a need in the art for a safe, effective treatment for SSADH.

### SUMMARY OF THE INVENTION

Presented here are compositions and methods for treating SSADHD. The methods employ mRNA therapy and are safe and reliable. The therapy utilizes lipid nanoparticles (LNPs) and/or micelles and/or other polymer-based delivery technologies to deliver the mRNA. In some aspects, the delivery technology targets the liver. However, delivery to the brain was also surprisingly observed, thereby providing significant metabolic improvement in the brain as well, without the disadvantages of ERT and AV-mediated therapy. For example, the benefits are not confined only to circulating enzyme levels and gene dose titration since repeated administration at different maturational ages is encompassed.

In an example embodiment, an SSADHD treatment dose of mRNA includes hALDH5A1 in a delivery medium such as lipid nanoparticles (LNPs) and/or micelles, independently or in a combination, optimized for therapeutic benefit. The mRNA encoding SSADH can alternately be an hALDH5A1 modified to be self-replicating.

In another variation, a method to treat SSADHD involves administering doses of mRNA composition to patients diagnosed with SSADHD, such as through intravascular or intrathecal injection, nasal delivery, intraperitoneal delivery, or oral delivery with acid-resistant delivery media such as pH sensitive micelles.

Accordingly, employing mRNA to restore normal SSADH activity, patients can move beyond less effective and higher risk symptomatic interventions and enjoy improved quality of life. This disclosure thus provides a combination of steps and substances not found in nature to reduce adverse health impacts of SSADHD.

It is an object of this disclosure to provide a method for treating succinic semialdehyde dehydrogenase deficiency (SSADHD) in a subject in need thereof, comprising administering to the subject a therapeutically effective amount of an mRNA coding for aldehyde dehydrogenase 5 family member A1 (ALDH5A1). In some aspects, the mRNA is administered by one or more routes selected from the group consisting of intravascular injection, intrathecal injection, nasal inhalation, pulmonary delivery, intraperitoneal delivery, sub-cutaneous delivery, and oral delivery. In other aspects, the mRNA is encapsulated in a delivery medium. In yet further aspects, the delivery medium comprises a plurality of micelles. In additional aspects, the micelles are pH sensitive micelles. In other aspects, the plurality of micelles comprises one or more self-assembling amphiphilic pH-sensitive copolymers containing hyaluronic acid, alginic acid, heparin, esters, acrylates, amino esters, carboxymethyl cellulose, carboxymethyl dextran, poly histidine, poly-vinyl pyridine, hydroxyethyl methacrylate, chitosan, tertiary amine starch, imine, and/or hydrazones. In further aspects, the delivery medium comprises one or more of a plurality of phospholipids, fatty acids, or other amphiphiles. In additional aspects, the delivery medium comprises lipid nanoparticles (LNPs). In other aspects, a sequence of the mRNA is at least 95% identical to SEQ ID NO: 1. In additional aspects, the mRNA codes for a c.354G>C variant of ALDH5A1. In some aspects the mRNA is modified to be self-replicating. In additional aspects, the step of administering delivers the mRNA to the brain of the subject.

### BRIEF DESCRIPTION OF THE FIGURES

FIG. 1 illustrates the disruption of GABA metabolism in SSADHD, including: glutamic acid decarboxylase (GAD); γ-aminobutyric acid (GABA); γ-hydroxybutyrate (GHB); GABA transaminase (GABA-T); succinic semialdehyde reductase (SSR); γ-hydroxybutyrate dehydrogenase (GHBDH); vigabatrin (VGB). SSADHD defect is indicated by X shows. Arrows (1/1) show increased/decreased metabolite levels in tissues and physiological fluids. GHB and GABA are increased in physiological fluids.

FIG. 2 Human ALDH5A1 protein expression and mitochondrial localization in ALDH5A1-mRNA-transfected (top row), eGFP mRNA (control mRNA; middle row) or untransfected SSADHD patient skin fibroblast. TOM20, mitochondrial marker; nuclei stained with DAPI. Representative images using confocal microscopy and Alexa-Fluor antibodies (Alexa Fluor®546/ALDH5A1, Alexa Fluor®647/TOM20; Santa Cruz). Cells were transfected with 500 ng mRNA using Lipofectamine™ MessengerMAX™ (Thermofisher). The figure shows successful mitochondrial expression of immunoreactive human ALDH5A1 (top row) in patients' skin fibroblasts transfected with codon-modified (TriLink®) human ADLH5A1 mRNA. Neither untransfected fibroblasts nor fibroblasts transfected with a sham mRNA (cGFP mRNA) show the presence of immunoreactive ALDH5A1.

FIG. 3A-C. (A) depicts ALDH5A1 activity in SSADHD patient and aldh5a1^{-/-} fibroblast cell lines 24 hrs after hALDH5A1- or cGFP-mRNA transfection. Activity was measured in cell homogenates by NADH fluorimetry and showed significant expression of enzymatically active ALDH5A1. (B) and (C) depict metabolic rescue in the same cell lines, showing improvement (decrease) in GHB (B) and GABA (C)) medium concentrations (ELISA). All data points are average of triplicate measurements. 0016-01, 005-01, 003-04, 003-01, 0024-02 are human skin fibroblast cell lines from individuals with genetically confirmed SSADH deficiency. Aldh5a1^{-/-} are aldh5a1-deficient mouse lung fibroblasts, Aldh5a1^{+/+} are wild-type mouse lung fibroblasts.

FIG. 4 shows in vivo studies. Shown are ALDH5A1 protein expression and activity in liver (left panels) and brain (right panels) of aldh5a1^{-/-} mice intravenously injected with either 1 mg/kg hALDH5A1-LNPs (Mut-mRNA) or PBS (Mut-PBS), and from a wild-type (WT) mouse (representative from 10-μm tissue sections; 10× magnification, confocal microscopy). Tissue collection was performed 48 hours after injection. Immunohistochemistry (IHC) (Protein) was performed with Alexa-Fluor anti-ALDH5A1 and anti-TOM20 antibodies with DAPI for nuclear staining. Note the positive ALDH5A1 staining in the liver and brain sections from mRNA-LNP-treated tissues, the absence of ALDH5A1 in the untreated aldh5a1^{-/-} sections, and the strong, expected staining in the wild-type sample. Enzyme histochemistry (EHC) (Activity) was performed using MTT. Note the positive, cell-specific expression of active ALDH5A1 in both the hALDH5A1-LNP treated aldh5a1^{-/-} brain sample and the wild-type specimen. Also note the denser staining in the mRNA-treated aldh5a1^{-/-} brain, suggestive of ALDH5A1 expression in reactive astrocytes.

FIG. 5 shows firefly luciferase activity in the gut and liver of aldh5a1^{-/-} mice 24 hours after i.p. injection of Fluc-LNPs (1 mg mRNA/kg). Images were taken ˜20 min. (whole mouse) and ˜30 min. (organs) after i.p. injection of 0.5 mg/kg luciferin (substrate for luciferase). Luminescence was detected using the IVIS® Spectrum In Vivo Imaging System. Fluc-mRNA (TriLink) was encapsulated in the same lipid nanoparticles as those used to deliver hALDH5A1 mRNA. This demonstrates that intraperitoneal injections of hALDH5A1 mRNA-LNPs will result in the expression of functional human ALDH5A1 in both liver and intestine.

FIG. 6A-C shows successful and prolonged in vitro rescue of the molecular (SSADH protein expression) and metabolic GABA phenotype of induced pluripotent stem cell (iPSC)-derived SSADHD neurons transfected with human ALDH5A1 mRNA. Two iPSC-derived neuronal cell lines were created: one from a carrier (Father) and one from his child with genetically confirmed diagnosis of SSADHD (Proband). The cells were studied either without transfection (Proband^{UNT}) or after transfection with hALDH5A1 mRNA (hALDH5A1 mRNA transfection using Lipofectamine™ MessengerMAX™ Transfection Reagent; Proband^{hALDHSA1}). As can be seen in (A), SSADH (ALDH5A1) protein expression (measured by Western blot as SSADH/β-actin ratio) was not detectable in Proband^{UNT} cells transfection but was induced in transfected neurons (p<0.013 for Proband^{UNT} vs. Proband^{hALDH51}) to levels not significantly different from those of the Carrier (Father) cells (NS). Normalization of SSADH expression lasted at least 7 days after transfection (B). Transfection also normalized GABA levels (expressed as nmoles/[SSADH/β-actin]ratios) in culture media, consistent with significant expression of immunoreactive and functional SSADH in proband-derived cells compared to those observed in Carrier (Father-derived) cells, indicating successful phenotypic rescue that lasts at least 7 days in vitro (C).

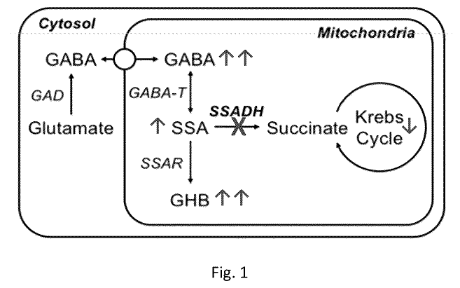

### DETAILED DESCRIPTION OF THE INVENTION

In the description herein, a word appearing in the singular encompasses its plural counterpart, and a word appearing in the plural encompasses its singular counterpart, unless implicitly or explicitly understood or stated otherwise. Furthermore, it is understood that for any given component or embodiment, any of the possible candidates or alternatives listed for that component may generally be used individually or in combination with one another, unless implicitly or explicitly understood or stated otherwise. Moreover, the figures are not necessarily drawn to scale, wherein some of the elements may be drawn merely for clarity. Also, reference numerals may be repeated among the various figures to show corresponding or analogous elements. Additionally, any list of such candidates or alternatives is merely illustrative, not limiting, unless implicitly or explicitly understood or stated otherwise. In addition, unless otherwise indicated, numbers expressing quantities of ingredients, constituents, reaction conditions and so forth used in the specification and claims are to be understood as being modified by the term “about.”

Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the standard deviation found in their respective testing measurements.

Various embodiments are described herein. In the following description, specific details of methods, organisms, systems, components, and operations are included to provide a thorough understanding of certain embodiments of the disclosed technology. A person skilled in the relevant art will also understand that the technology may have additional embodiments. The technology may also be practiced without several of the details of the embodiments described below.

### Definitions

A lipid is any of a large group of organic compounds that are oily to the touch and insoluble in water. Lipids include fatty acids, oils, waxes, sterols, and triglycerides.

Phospholipid: A phospholipid is a molecule with two fatty acids and a modified phosphate group attached to a glycerol backbone, i.e. a phospholipid has a hydrophilic “head” containing a phosphate group and two hydrophobic “tails” derived from fatty acids, joined by an alcohol residue (usually a glycerol molecule). The phosphate group may be modified by the addition of charged or polar chemical groups, e.g. choline, serine, etc.

As used herein, a fatty acid is a carboxylic acid with an aliphatic chain, which is either saturated or unsaturated. Most naturally occurring fatty acids have an unbranched chain of an even number of carbon atoms, from 4 to 28 and may exist as three main classes of esters: triglycerides, phospholipids, and cholesteryl esters. Short-chain fatty acids (SCFAs) are fatty acids with aliphatic tails of five or fewer carbons (e.g. butyric acid). Medium-chain fatty acids (MCFAs) are fatty acids with aliphatic tails of 6 to 12 carbons, which can form medium-chain triglycerides. Long-chain fatty acids (LCFAs) are fatty acids with aliphatic tails of 13 to 21 carbons. Very long chain fatty acids (VLCFAs) are fatty acids with aliphatic tails of 22 or more carbons. Saturated fatty acids have no C═C double bonds and have the formula CH₃ (CH₂)nCOOH, for different n. Unsaturated fatty acids have one or more C═C double bonds. The C═C double bonds can give either cis or trans isomers.

An amphiphile or amphipath is a chemical compound possessing both hydrophilic (water-loving, polar) and lipophilic (fat-loving) properties. Amphiphilic compounds include surfactants and detergents. The phospholipid amphiphiles are the major structural component of cell membranes.

A nanoparticle or ultrafine particle is a particle of matter 1 to 100 nanometers (nm) in diameter. The term is sometimes used for larger particles, up to 500 nm or fibers and tubes that are less than 100 nm in only two directions. If one of the characteristic dimension is in the nano range (1-100 nm) the particle can be classified as a nanoparticle, even if its other dimensions are outside that range. Nanoparticles are distinguished from microparticles (1-1000 μm), “fine particles” (sized between 100 and 2500 nm), and “coarse particles” (ranging from 2500 to 10,000 nm). In some aspects, nanoparticles encompass, for example, micelles and lipid nanoparticles.

Lipid nanoparticles (LNPs) are nanoparticles composed of lipids. A lipid nanoparticle (LNP) is typically spherical with an average diameter between 10 and 1000 nanometers. Solid lipid nanoparticles possess a solid lipid core matrix that can solubilize lipophilic molecules. The lipid core is typically stabilized by surfactants (emulsifiers). Lipids include triglycerides (e.g. tristearin), diglycerides (e.g. glycerol behenate), monoglycerides (e.g. glycerol monostearate), fatty acids (e.g. stearic acid), steroids (e.g. cholesterol), and waxes (e.g., cetyl palmitate). All classes of emulsifiers (with respect to charge and molecular weight) have been used to stabilize the lipid dispersion. In particular, cholesterol is important for organ targeting (Su, K., et al. Nat Commun 15, 5659 (2024)).

A micelle is a self-assembled aggregate of amphiphilic macromolecules (or supramolecular assembly) or self-assembly of surfactant amphipathic lipid molecules dispersed in a liquid, forming a colloidal suspension (also known as associated colloidal system). A typical micelle in water forms an aggregate with the hydrophilic “head” regions in contact with surrounding solvent, sequestering the hydrophobic single-tail regions in the micelle center or core of micelle. This phase is caused by the packing behavior of single-tail lipids in a bilayer. Spherical micelles tend to range in size from 10 to 100 nm in diameter (polymeric micelles can exceed the common size range of 10 to 100 nm depending on the structure of copolymers) For example, see Masayuki Yokoyama et. al., Journal of Controlled Release Volume 55, Issues 2-3, 13 Nov. 1998, Pages 219-229; Cabral et. al., Nat. Nanotechnol. 2011, 6, 815-823; and Anouk Lie-Pianget, al., Food Research International, Volume 139, January 2021, 109939. However, they can become larger when cargos are encapsulated within their hydrophobic cores. pH-Sensitive micelles typically are formed from components which have the ability to aggregate and form micelles at certain pH but which disaggregate (disassemble) at a specific pH of interest.

Lamellae particles comprise a regular alternation of amorphous and crystalline phases stacked in parallel sheets. Lamellae particle may or may not contain lipids.

Exosomes are extracellular vesicles generated by all cells and. However, artificial exosomes can also be created. Exosomes are naturally occurring membrane-bound extracellular vesicles (EVs) ranging in size from 30 to 150 nanometers. They carry nucleic acids, proteins, lipids, and metabolites and function as mediators of near and long-distance intercellular communication in health and disease. However, artificial exosomes can also be produced (engineered) synthetically for use in exosome platform-based therapeutics for disease management. See, for example, published United States patent application US20230181466 and issued US patents U.S. Pat. Nos. 11,938,219 and 10,590,417, the complete contents of each of which is hereby incorporated by reference in entirety.

Dendrimers are synthetic polymers with a structure of repeatedly branching chains, typically forming spherical nano-sized, radially symmetric molecules with a well-defined, homogeneous, and monodisperse structure that comprises an internal cavity or void space. Dendrimers typically have a symmetric core, an inner shell, and an outer shell.

Polymersomes, composed of amphiphilic block copolymers, are stable, self-assembled vesicles comprising an aqueous interior that is separated from surrounding media by an amphiphilic polymer bilayer. The thickness of the bilayer (5-30 nm) usually causes a robust and impermeable wall.

Poly(β-amino ester)s (PβAEs) are biodegradable cationic polymers that are based on poly(amidoamine) s and contain tertiary amines in their backbones. PBAEs can be synthesized by a Michael addition reaction. Depending on the use of a monomer in stoichiometric excess, PβAEs can have either amine- or diacrylate-terminated chains. Various types are known, for example, homopolymer PβAEs, star-shaped PβAEs, branched and hyperbranched PβAEs, and various hybrid nanovectors based on PBAEs complexed with lipids, commonly known as lipopolyplexes. See also issued US patents U.S. Pat. Nos. 8,562,966, 8,808,681 and 11,136,597 and published US patent application US20200297851, the complete contents of each of which is hereby incorporated by reference in entirety.

The gut is another term for the gastrointestinal tract. The portions of the alimentary canal, particularly the stomach and the intestines.

The Enzyme and HALDH5A1 mRNA Encoding the Enzyme

Fully or partially restoring normal SSADH activity in an SSADHD patient in need thereof is accomplished by administering to the patient mRNA coding for an active form of SSADH, thereby providing significant neurometabolic improvement/therapeutic benefit. Specifically, RNA encoding hALDH5A1 is formulated or compounded and administered to patients via an effective delivery medium, such as encapsulation within lipid nanoparticles (LNPs) and/or via micelles.

As used herein, “SSADH” refers to aldehyde dehydrogenase 5a1, ALDH5A1; E.C. 1.2.1.24; OMIM 610045, 271980. The present disclosure encompasses various nucleic acids, generally synthetic, genetically engineered mRNA, which are administered to a subject in need thereof, e.g. a subject suffering from a deficit of SSADH activity, in order to provide to the subject a form of the SSADH enzyme that exhibits sufficient in vivo activity to prevent and/or treat the deficiency.

Those of skill in the art are well-acquainted with protocols for making/manufacturing synthetic mRNA. For example, mRNA can be synthesized in a cell-free system via a process called in vitro transcription (IVT). Any method or technique known in the art may be used to prepare suitable mRNA, as long as the resulting mRNA is sufficiently stable to be compounded into a composition suitable for administration to a subject and to undergo translation into a biologically (physiologically) active SSADH in vivo.

The nucleic acid sequence of an exemplary full-length ALDH5A1 mRNA that encodes a biologically functional SSADH protein is shown in SEQ ID NO: 1.

Transcript ID: ENST00000357578.8 (Name: ALDH5A1-202; 1608 nt; 5131 base pairs)

(SEQ ID NO: 1)
ATGGCGACCTGCATTTGGCTGCGGAGCTGTGGGGCCCGGCGCCTCGGGTC
GACGTTTCCAGGCTGCCGCCTCCGCCCCCGCGCCGGCGGCCTGGTCCCTG
CCTCCGGGCCTGCGCCCGGCCCGGCCCAGCTCCGCTGCTACGCTGGGCGC
CTGGCGGGCCTCTCTGCGGCGCTGCTGCGCACCGACAGCTTCGTGGGCGG
CCGCTGGCTCCCGGCCGCCGCCACCTTCCCCGTGCAAGACCCGGCCAGCG
GCGCCGCTCTGGGCATGGTAGCCGACTGCGGGGTGCGAGAGGCCCGCGCC
GCCGTGCGCGCTGCCTACGAGGCTTTCTGCCGCTGGAGGGAGGTCTCCGC
CAAGGAGAGGAGTTCATTACTTCGGAAGTGGTACAATTTAATGATACAAA
ATAAGGATGACCTTGCCAGAATAATCACAGCTGAAAGTGGAAAGCCACTG
AAGGAGGCACATGGAGAAATTCTCTATTCCGCCTTTTTCCTAGAGTGGTT
CTCTGAGGAAGCCCGCCGTGTTTACGGAGACATTATCCACACCCCGGCAA
AGGACAGGCGGGCCCTGGTCCTCAAGCAGCCCATAGGCGTGGCTGCAGTC
ATCACCCCGTGGAATTTCCCCAGTGCCATGATCACCCGGAAGGTGGGGGC
CGCCCTGGCAGCCGGCTGTACTGTCGTGGTGAAGCCTGCCGAAGACACGC
CCTTCTCCGCCCTGGCCCTGGCTGAGCTTGCAAGCCAGGCTGGGATTCCT
TCAGGTGTATACAATGTTATTCCCTGTTCTCGAAAGAATGCCAAGGAAGT
AGGGGAGGCAATTTGTACTGATCCTCTGGTGTCCAAAATTTCCTTTACTG
GTTCAACAACTACAGGAAAGATCCTGTTGCACCACGCAGCAAACTCTGTG
AAAAGGGTCTCTATGGAGCTGGGCGGCCTTGCTCCATTTATAGTATTTGA
CAGTGCCAACGTGGACCAGGCTGTAGCAGGGGCCATGGCATCTAAATTTA
GGAACACTGGACAGACTTGTGTTTGCTCAAACCAATTCTTGGTGCAAAGG
GGCATCCATGATGCCTTTGTAAAAGCATTCGCCGAGGCCATGAAGAAGAA
CCTGCGCGTAGGTAATGGATTTGAGGAAGGAACTACTCAGGGCCCATTAA
TTAATGAAAAAGCGGTAGAAAAGGTGGAGAAACAGGTGAATGATGCCGTT
TCTAAAGGTGCCACCGTTGTGACAGGTGGAAAACGACACCAACTTGGAAA
AAATTTCTTTGAGCCTACCCTGCTGTGCAATGTCACCCAGGACATGCTGT
GCACTCATGAAGAGACTTTCGGGCCTCTGGCACCAGTTATCAAGTTCGAT
ACAGAGGAGGAGGCTATAGCAATCGCTAACGCAGCTGATGTTGGGTTAGC
AGGTTATTTTTACTCTCAAGACCCAGCCCAGATCTGGAGAGTGGCAGAGC
AGCTGGAAGTGGGCATGGTTGGCGTCAACGAAGGATTAATTTCCTCTGTG
GAGTGCCCTTTTGGTGGAGTGAAGCAGTCCGGCCTTGGGCGAGAGGGGTC
CAAGTATGGCATTGATGAGTATCTGGAACTCAAGTATGTGTGTTACGGGG
GCTTGTAG

This mRNA was synthesized and utilized in the Examples presented herein.

The amino acid sequence of the exemplary SSADH protein that is encoded by SEQ ID NO: 1 (and which is biologically active when translated in vivo as described herein) is shown in SEQ ID NO: 2.

Protein sequence: 535aa (see the website located at useast.ensembl.org/Homo_sapiens/Transcript/ProteinSummary?db=core; g=ENSG00000112294; r=6:24494867-24537207; t=ENST00000357578).

### Length: 535; Mass (Da); 57,215;

(SEQ ID NO: 2)
MATCIWLRSC GARRLGSTFP GCRLRPRAGG LVPASGPAPG
PAQLRCYAGR LAGLSAALLR TDSFVGGRWL PAAATFPVQD
PASGAALGMV ADCGVREARA AVRAAYEAFC RWREVSAKER
SSLLRKWYNL MIQNKDDLAR IITAESGKPL KEAHGEILYS
AFFLEWFSEE ARRVYGDIIH TPAKDRRALV LKQPIGVAAV
ITPWNFPSAM ITRKVGAALA AGCTVVVKPA EDTPFSALAL
AELASQAGIP SGVYNVIPCS RKNAKEVGEA ICTDPLVSKI
SFTGSTTTGK ILLHHAANSV KRVSMELGGL APFIVFDSAN
VDQAVAGAMA SKFRNTGQTC VCSNQFLVQR GIHDAFVKAF
AEAMKKNLRV GNGFEEGTTQ GPLINEKAVE KVEKQVNDAV
SKGATVVTGG KRHQLGKNFF EPTLLCNVTQ DMLCTHEETF
GPLAPVIKFD TEEEAIAIAN AADVGLAGYF YSQDPAQIWR
VAEQLEVGMV GVNEGLISSV ECPFGGVKQS GLGREGSKYG
IDEYLELKYV CYGGL

Those of skill in the art will recognize that, due to the redundancy (degeneracy) of the genetic code, certain modifications to the sequence of SEQ ID NO: 1 can be made and still result in the translation of a protein that is identical to SEQ ID NO: 2. Redundancy in the genetic code means that most amino acids are specified by more than one mRNA codon. For example, the amino acid phenylalanine (Phe) is specified by the codons UUU and UUC; the amino acid glutamic acid is specified by GAA and GAG codons (difference in the third position); the amino acid leucine is specified by UUA, UUG, CUU, CUC, CUA, CUG codons (difference in the first or third position); and the amino acid serine is specified by UCA, UCG, UCC, UCU, AGU, AGC (difference in the first, second, or third position), as shown in the standard RNA codon table shown below:

| Amino | | |
|---|---|---|
| acid | RNA codons | Compressed |
| Ala, A | GCU, GCC, GCA, | GCN |
| | GCG | |
| Arg, R | CGU, CGC, CGA, | CGN, AGR; |
| | CGG; AGA, AGG | or |
| | | CGY, MGR |
| Asn, N | AAU, AAC | AAY |
| Asp, D | GAU, GAC | GAY |
| Asn or | AAU, AAC; GAU, | RAY |
| Asp, B | GAC | |
| Cys, C | UGU, UGC | UGY |
| Gln, Q | CAA, CAG | CAR |
| Glu, E | GAA, GAG | GAR |
| Gln or | CAA, CAG; GAA, | SAR |
| Glu, Z | GAG | |
| Gly, G | GGU, GGC, GGA, | GGN |
| | GGG | |
| His, H | CAU, CAC | CAY |
| START | AUG, CUG, UUG | HUG |
| Ile, I | AUU, AUC, AUA | AUH |
| Leu, L | CUU, CUC, CUA, | CUN, UUR; |
| | CUG; UUA, UUG | or |
| | | CUY, YUR |
| Lys, K | AAA, AAG | AAR |
| Met, M | AUG | |
| Phe, F | UUU, UUC | UUY |
| Pro, P | CCU, CCC, CCA, | CCN |
| | CCG | |
| Ser, S | UCU, UCC, UCA, | UCN, AGY |
| | UCG; AGU, AGC | |
| Thr, T | ACU, ACC, ACA, | ACN |
| | ACG | |
| Trp, W | UGG | |
| Tyr, Y | UAU, UAC | UAY |
| Val, V | GUU, GUC, GUA, | GUN |
| | GUG | |
| STOP | UAA, UGA, UAG | URA, UAG; |
| | | or |
| | | UGA, UAR |

In some aspects, the synthetic (engineered) in vitro mRNA is chemically modified to ensure greater in vivo stability and/or transcription efficiency, and/or for another reason, (e.g. to include an enzymatic cleavage site or a site for in vitro modification) compared to naturally occurring RNA or unmodified synthetic RNA or SEQ ID NO: 1, but without interfering with its in vivo translational function. Examples of RNA modifying techniques include but are not limited to: various capping technologies, either post-transcriptionally using capping enzymes such as Vaccina Capping Enzyme (VCE), the Faustovirus Capping Enzyme, or mRNA cap 2′-O-methyltransferase; or co-transcriptionally by including synthetic RNA cap analogs including 2′ O-methylation on the first base, 3′ O-methylation on m7G, an m6A modification, or the alphavirus 5′ cap, e.g. using the trinucleotide CleanCap® reagent AG, etc. A 5′-CAP is typically a modified nucleotide, in particular a guanine nucleotide, added to the 5 ‘end of an mRNA molecule. Preferably, the 5’-CAP is added using a 5′-5′-triphosphate linkage (also called m7GpppN). Other examples of 5′-CAP structures include glyceryl, inverted deoxy abasic residue (portion), 4 ‘nucleotides, 5’-methylene, 1-(beta-D-erythrofuranosyl) nucleotide, 4′-thio nucleotides, carbocyclic nucleotides, nucleotide 1, 5-anhydrohexitol, L-nucleotides, alpha-nucleotide, modified base nucleotides, threo-pentofuranosyl nucleotide, acyclic 3 ‘, 4’-seco nucleotides, acyclic 3,4-dihydroxybutyl nucleotides, acyclic 3,5-dihydroxypentyl nucleotides, acyclic residues 3′-3′-inverted nucleotide, 3′-3′-inverted abasic residues, 3′-2′-inverted nucleotide residues and 3′-2′-inverted abasic residues, 1,4-butanediol phosphate, 3 ‘-phosphoramidate, hexylphosphate, aminohexylphosphate, 3’-phosphate, 3′-phosphorothioate, phosphorodithioate or binding or non-binding methylphosphonate residues. These modified 5′-CAP structures can be used in the context of the present disclosure to modify the mRNA sequence. Additional modified 5′-CAP structures that can be used in the context of the present disclosure are CAP1 (ribose methylation of the adjacent nucleotide of m7GpppN), CAP2 (ribose methylation of the second nucleotide downstream of m7GpppN), CAP3 (ribose methylation of the third nucleotide downstream of m7GpppN), CAP4 (ribose methylation of the fourth nucleotide downstream of m7GpppN), ARCA (anti-reverse CAP analog), modified ARCA (e.g. phosphothioate modified ARCA), inosine, N1-methylguanosine, 2′-fluoroguanosine, 7-deazaguanosine, 8-oxoguanosine, 2-aminoguanosine, LNA-guanosine and 2-azidoguanosine.

The use of at least one modified nucleoside such as pseudouridine (ψ), 5-methylcytosine (m⁵C), 5-methyluridine (m⁵U), 2′-O-methyluridine (Um or m^{2′O}U), 2-thiouridine (s²U), phosphorothioate nucleotide, 2′-deoxyfluoro ribose nucleotide, and N′-methyladenosine (m⁶A) in place of at least a portion of the corresponding unmodified canonical nucleoside e.g., in place of one or more of the corresponding unmodified A, C, G, or T canonical nucleosides.

In addition, the inclusion of a poly A tail on the RNA is contemplated. A poly-A tail makes the RNA molecule more stable and prevents its degradation, e.g. in vivo. Additionally, the poly-A tail allows mature messenger RNA molecule to be exported from the nucleus and translated into a protein by ribosomes in the cytoplasm. Generally, the length of a poly A tail is from about 100 to about 250 residues, such as about 100, 125, 150, 175, 200, 225 or 250 residues.

In some aspects of this disclosure, TriLink® technology is used to manufacture the mRNA. This technology uses a specially designed plasmid with all the required elements for transcription (a T7 promoter and proprietary 5′ and 3′ UTRs), and it is designed to incorporate a 120 nt poly A tail via PCR.

In some aspects, the mRNA is modified to be more lipophilic without interfering with translation, e.g. by conjugation to lipophiles such as cholesterol, α-tocopherol, trolox, palmitate, stearate, distearoyl-lipid, ibuprofen, etc.; and other modifications known in the art, e.g. see the article linked to the website located at mdpi.com/1420-3049/29/2/452, the complete contents of each of which are hereby incorporated by reference in entirety.

In some aspects, the mRNA contains more than one modification. For example, one or more phosphate modifications in combination with at least one lipid (dodecyl and cholesterol) modifications can be used.

In some aspects, the mRNA is modified by codon optimization for expression in humans. Codon optimization is a process used to improve gene expression and increase the translational efficiency of a gene of interest, e.g. by accommodating codon bias of a host organism, to increase mRNA stability and/or translation efficiency (see, for example, Presnyak, V. et al. Codon optimality is a major determinant of mRNA stability. Cell 160, 1111-1124 (2015)), or for expression in a particular organ or tissue, etc. or for any other reason that serves to increase and/or maintain a suitable level of expression of the mRNA at a desired location or locations.

In further aspects, the mRNA is modified to be self-replicating (self-amplifying) such as those used to develop vaccines. Self-amplifying RNA (saRNA)-based nucleic acids have the advantage of requiring much lower doses than conventional mRNA to obtain similar levels of protein expression. saRNA vaccines are generally derived from the genomes of RNA viruses, such as alphaviruses, flaviviruses, and rhabdoviruses, among others. For example, an alphavirus saRNA has two open reading frames (ORFs), one coding for the viral replicase (Rep) and a second one coding for a protein of interest such as SSADH or a variant thereof as discussed herein. Further, see Saraf, A., et al. Nat Med 30, 1363-1372 (2024); Silva-Pilipich, et al. Vaccines 2024, 12, 318; and the article found at the website located at cell.com/trends/biotechnology/fulltext/S0167-7799 (23) 00154-3, the complete contents of each of which are hereby incorporated by reference in entirety.

In some aspects, the mRNA is modified from the SEQ ID NO: 1 sequence in order to encode an alternative form of the encoded SSADH protein. Those of skill in the art will recognize that various amino acid modifications, such as amino acid substitutions, may be made in the encoded SSADH protein sequence without compromising or decreasing the ability of the protein to perform its desired function, i.e. to function as a biologically active SSADH. Suitable encoded sequences are typically substantially identical to SEQ ID NO: 2, i.e. they are at least 80%, 85%, 90%, 92%, 95%, 97%, 98%, 99% or more identical to SEQ ID NO: 2.

“Modifications” as used herein with respect to the sequence of SEQ ID NO:2 (or with respect to other sequences) refer to the corresponding amino acid positions of SEQ ID NO:2 and include sequence substitutions, deletions, insertions and/or additions. Such modifications do not decrease the biological activity of the protein, or at least do not decrease the activity by more than 25%, 20%, 15%, 10%, or 5% or less, as measured by a standard, art-accepted technique. Such decreases may be acceptable if other advantages accrue, such as increased stability, longevity, etc. Preferably, the modification increases activity of the enzyme, e.g. by at least 5, 10, 15, 20, 25, 30, 35, 40, 45, 50, 55, 60, 65, 70 or even 75% or more activity, when measured using standard enzyme assays known in the art for this enzyme.

One caveat limitation in using self-amplifying mRNA to rescue a gene defect is uncontrolled and overexpression expression of the protein enzyme is to be avoided. Thus, in some aspects, the ALDH5A1 mRNA and/or the self-amplifying ALDH5A/mRNA also contains a suitable regulatory component of the self-amplification process, providing fine titration of protein expression. Such regulatory elements are known in the art.

Measurement of ALDH5A1 enzymatic activity is performed using a fluorometric assay designed to quantify the generation of NADH from NAD+ in the presence of succinate semialdehyde (SSA) consumption, as previously described (Pop A, et al. Mol. Genet. Metab., 2020; 130:172-178). In a typical example, samples (e.g. cell or tissue homogenates) are assayed in triplicate, with and without 0.1-2 mM succinic semialdehyde (SSA) as substrate. Incubations are performed for 90 min at 37° C., using 3-20 ug of sample protein in a 150-μL reaction mixture containing 90 mmol/L Tris-HCl buffer pH=8.6, 0.9 mmol/L EDTA, 45 mmol/L KCl, 18 mmol/L dithiothreitol, and 0.5 mmol/L NAD+. The incubation medium may also contain chemicals that will specifically inhibit other NAD-consuming enzymes present in the samples (e.g. malonic acid, a competitive inhibitor of succinate dehydrogenase), or chemicals that inhibit ALDH5A1 (e.g. para-hydroxybenzaldehyde) to ensure specificity of the assay for ALDH5A1. A NADH calibration curve, ranging from 0.01 to 0.33 mmol/L is performed for every experiment. NADH-specific fluorescence is measured at λₑₘ 460 nm with λₑₓ set at 350 nm using a SpectraMax® iD3 Microplate Reader. Enzyme activity is calculated from the slope of NADH production over time.

A “substitution” with respect to SEQ ID NO:2 (or with respect to another SSADH sequence disclosed herein) is a substitution of a particular amino acid residue at the corresponding amino acid position of SEQ ID NO:2 and refers to the exchange with a different amino acid residue.

In some aspects, the exchange is a “conservative” amino acid substitution. A conservative amino acid substitution is one in which one amino acid residue is replaced with another amino acid residue having a similar side chain. Families of amino acid residues with similar side chains include basic side chains (e.g. lysine (K), arginine (R), histidine (H)), acidic side chains (e.g. aspartic acid (D), glutamic acid (E)), uncharged polar side chains (e.g. glycine (G), asparagine (N), glutamine (Q), serine(S), threonine (T), tyrosine (Y), cysteine (C)), non-polar side chains (e.g. alanine (A), valine (V), leucine (L), isoleucine (I), proline (P), phenylalanine (F), methionine (M), tryptophan (W), β-branched side chains (e.g., threonine (T), valine (V), isoleucine (I)), and aromatic side chains (e.g., tyrosine (Y), phenylalanine (F), tryptophan (W), histidine (H)). are defined in the art. For example, substitution of phenylalanine for tyrosine is a conservative substitution. Methods for identifying conservative and non-conservative nucleotide and amino acid substitutions that confer, alter, or maintain a desired biological activity are known in the art (e.g., Brummell, Biochem. 32:1180-1187 (1993); Kobayashi, Protein Eng. 12 (10): 879-884 (1999); and Burks, PNAS 94:412-417 (1997)).

In other aspects, the change is a “non-conservative” amino acid substitution. A “non-conservative” amino acid substitution is one in which one amino acid residue is replaced with another amino acid residue having a different type of side chain, such as the exchange of a charged side chain with an aliphatic side chain or vice versa, or the exchange of a positively charged side chain for a negatively charged side change, etc.

In some aspects, a non-standard amino acid is present in the protein. However, in this case, the non-standard amino acid (either uncommon and not included in the standard 20 canonical amino acids, or altogether unnatural, i.e. not occurring in nature) must not decrease the biological activity of the protein as discussed above and must be encoded by the RNA that is administered. For example, UGA can code for selenocysteine and UAG can code for pyrrolysine.

In some aspects, the SSADH enzyme is the c.354G>C variant (Lys118Asn), a variant reported to have significantly higher (˜165%) enzyme activity than the SSADFH enzyme having SEQ ID NO: 2. The amino acid sequence of the c.354G>C is presented in SEQ ID NO:3 and the mRNA encoding this form of SSADH is presented in SEQ ID NO: 4. In particular embodiments, the version of SSADH is codon-optimized c.354G>C.

### Pharmaceutical Compositions

The mRNAs described herein are generally delivered (administered) in a polymer-based nanoparticle, examples of which include lipid-based carriers (such as lipid nanoparticles (LNPs), micelles, liposomes, and exosomes), etc., as well as block co-polymer-based polymersomes; lamella particles; dendrimers, and hybrids thereof, e.g. dendrimer LNPs; and poly(beta-amino ester) s (PBAE) s and lipid hybrids thereof.

### LNPs in General

Lipid nanoparticles (LNPs) are spherical vesicles that are made up of both solid and liquid lipids and stabilized by emulsifiers and are used as a carrier molecule for therapeutic, gene, or drug delivery. LNPs are thus nano-sized colloidal drug delivery systems that contain a lipid mixture (mixed amphiphiles) comprising both solid and liquid lipids in their core. Exemplary LNPs used, for example, in mRNA vaccines, are made of four types of lipids: an ionizable cationic lipid (whose positive charge binds to negatively charged mRNA), a PEGylated lipid (for stability), a phospholipid (for structure), and cholesterol (for structure). However, because of rapid clearance by the immune system of the positively charged lipid, neutral ionizable amino lipids have been developed and may be preferred. A novel squaramide lipid (that is, partially aromatic four-membered rings, which can participate in pi-pi interactions) is utilized in some delivery systems. LNPs formulated with such ionizable cationic lipids typically enter cells through receptor-mediated endocytosis and end up inside endosomes. The acidity inside the endosomes causes LNPs' ionizable cationic lipids to acquire a positive charge, and this is thought to allow LNPs to escape from endosomes and release their RNA payloads. Methods of making LNPs and are disclosed, for example, in published United States patent applications US20210046192A1 and US20220009878A1 and in issued US patent applications U.S. Pat. No. 10,207,010B2 and U.S. Pat. No. 11,771,653B2, the complete contents of each of which is hereby incorporated by reference in entirety.

In some aspects, mRNA-containing hALDH5A1-LNPs are prepared using microfluidic mixing as known in the art. For example, lipids are dissolved in a suitable carrier (e.g. an alcohol such as ethanol) and mRNA is diluted in a suitable buffer (e.g. citrate buffer). The lipid and mRNA solutions are then mixed (at a ratio of e.g. about 1:3) and then diluted and/or concentrated and/or dialyzed and/or sized as needed to achieve desired LNP size distributions, zeta potentials, stability, etc. Issued US patents U.S. Pat. Nos. 9,758,795, 11,771,653 and 1,174,480 and published US patent applications US20210046192A1 and US20220062175A1, the entire contents of each of which are hereby incorporated by reference in entirety, also describe various methods of making LPNs suitable for use in the present methods.

Preferably, the LNPs have a narrow size distribution ranging from, for example, about 50 to 135, or 55 to 130, or 60 to 125, or 65 to 120, or 70 to 115, or 75 to 110, or from about 80 to 100 nm, or mixtures of these sizes. In some aspects, the size distribution is from about 80 to 100 nm.

The LNPs generally have a high mRNA encapsulation efficiency of at least about 50, 55, 60, 65, 70, 75, 80, 85, 90, or 95%, or even higher (>95%) such s 96, 97, 98 or 99%. In typical formulations, LNPs are stable≤about at least 1-10 weeks, such as ≤about 1, 2, 3, 4, 5, 6, 7, 8, 9, 10 or more weeks (e.g. about 1-6 months), preferably at least about 4 weeks, when stored e.g. at +4° C.

Nanoparticle size plays a role in LNP properties, influencing stability, biodistribution, cellular uptake, and overall therapeutic efficacy. As is recognized by those of skill in the art, smaller LNPs (size<100 nm) tend to exhibit enhanced cellular uptake and prolonged circulation time, while larger LNPs (size>100 nm) offer higher drug loading capacity but reduced cellular uptake. A highly positive or negative Zeta potential indicates a strong surface charge, which results in electrostatic repulsion between particles and enhancement of their stability in the dispersion. On the other hand, a Zeta potential close to zero suggests a low surface charge and a higher tendency for particle aggregation or coalescence, potentially leading to the destabilization of the LNP suspension.

Surface morphology assessment of LNPs can be performed using transmission electron microscopy (TEM), scanning electron microscopy (SEM), and atomic force microscopy (AFM). A well-defined and smooth surface morphology is highly advantageous for LNPs in biological environments as it contributes to enhanced stability and mitigates the risk of opsonization, where serum proteins bind to the surface, triggering immune system clearance. This smooth surface minimizes unwanted interactions with biological components, promoting favorable LNP biodistribution and ensuring optimal therapeutic efficacy. On the other hand, LNPs with rough or irregular surface morphology may exhibit increased interactions with biological entities, potentially influencing LNP biodistribution patterns and impacting overall therapeutic effectiveness.

LNP surface modification by PEGylation or targeting ligands can further influence the surface morphology of LNPs. For instance, these modifications can improve the stealth properties of LNPs, reducing recognition by the immune system and therefore extending drug circulation time for better drug delivery efficiency.

LNP stability, loading efficacy, and entrapment efficiency are factors in the development and optimization of LNP-based drug delivery systems. LNP stability refers to the capability of LNPs to preserve their physical and chemical properties over time under various conditions. These properties include shape, size, and lipid component integrity during long-term storage or exposure to different biological environments like blood, plasma, or varying pH conditions. LNP drug loading capacity quantifies the total amount of drugs that can be loaded into the delivery system, while entrapment efficiency measures the effectiveness of the formulation process in retaining drug within the LNP.

In some aspects, LNPs are subdivided into five subgroups: liposomes, lipid nano emulsions, solid lipid nanoparticles, nanostructured lipid carriers, and lipid-polymer hybrid nanoparticles, as described below. All these can be used either independently or in combination to deliver therapeutic or small molecules such as the miRNA described herein under controlled conditions.

Liposomes: Liposomes are spherical vesicles with a bilayer structure made of phospholipid molecules that separates an aqueous internal compartment from the bulk aqueous phase. Lipid nanoparticles usually only have a single phospholipid outer layer that encapsulates the interior, which may be non-aqueous. Liposome size range is up to about 500 nm. The ability of liposomes to encapsulate hydrophilic or lipophilic drugs have allowed these vesicles to become useful drug delivery systems. Liposomes are described in more detail below.

Lipid nano emulsions (LNE): Lipid nanosized emulsions are oil in water dispersions with an oil droplet size of about 200 nm. LNEs are commonly composed of oil such as medium-chain triglyceride, soybean oil, lecithin, are prepared by a solvent-diffusion method in an aqueous system. These are modified with polyethylene glycol (PEG) to control size and encapsulation ability of LNE.

Solid Lipid Nanoparticles (SLN): These are colloidal carriers which are submicron-sized lipid emulsions where the liquid lipid (oil) is substituted by a solid lipid. These then offer small size, large surface area, higher interaction of phases at the interfaces.

Nanostructured lipid carriers: A synergistic combination of liquid and solid lipids are use to develop molecules that can act as carrier molecules.

Lipid polymer hybrid nanoparticles: The nanostructures that are made up of a polymeric core surrounded by a lipid layer.

### Liposomes

In some aspects, liposomes may also be used to deliver the mRNA. A liposome is a small artificial vesicle, spherical in shape, having at least one lipid bilayer. Due to their hydrophobicity and/or hydrophilicity, biocompatibility, particle size and many other properties, liposomes can be used as drug delivery vehicles for administration of pharmaceutical drugs and nutrients, such as lipid nanoparticles in mRNA vaccines, and DNA vaccines.

Liposomes are most often composed of phospholipids, especially phosphatidylcholine, and cholesterol, but may also include other lipids, such as those found in egg and phosphatidylethanolamine, as long as they are compatible with lipid bilayer structure. A liposome design may employ surface ligands for attaching to desired cells or tissues.

Based on vesicle structure, there are seven main categories for liposomes: multilamellar large (MLV), oligolamellar (OLV), small unilamellar (SUV), medium-sized unilamellar (MUV), large unilamellar (LUV), giant unilamellar (GUV) and multivesicular vesicles (MVV). The major types of liposomes are the multilamellar vesicle (MLV, with several lamellar phase lipid bilayers), the small unilamellar liposome vesicle (SUV, with one lipid bilayer), the large unilamellar vesicle (LUV), and the cochleate vesicle. A less usual form is multivesicular liposomes in which one vesicle contains one or more smaller vesicles.

Further descriptions of liposomes are found, for example, in United States published patent application publication 2007/0042031 and US20230181567 and is issued US patents U.S. Pat. Nos. 9,394,234, 9,737,528, 10,016,389 and 10,722,467, the complete contents of each of which is hereby incorporated by reference in entirety.

### Other Carriers

Micelles are tiny particles made of surfactant molecules that form closed lipid monolayers with a fatty acid or a hydrophobic core and polar or hydrophilic surface, allowing them to remain dispersible in water. Polymeric micelles can exceed the common size range of 100 nm depending on the structure of the copolymers. Micelles may be formed from, for example, phospholipids, fatty acids, or other amphiphiles. Block copolymers are often used. For example, suitable micelles include but are not limited to self-assembling amphiphilic pH-sensitive copolymers containing hyaluronic acid, alginic acid, heparin, esters, acrylates, amino esters, carboxymethyl cellulose, carboxymethyl dextran, poly histidine, poly-vinyl pyridine, hydroxyethyl methacrylate, chitosan, tertiary amine starch, imine, hydrazones, and the like. Suitable micelles typically range size in average size from about 100 to 800 nm, such as about 100, 200, 300, 400, 500, 600, 700 or 800 nm, or mixtures of these sizes. Once they are loaded with a molecule of interest, such as mRNA as described herein, their size may expand significantly. They may be stable i.e. remain as micelles, at a pH of interest such as at physiologic or neutral pH and disassociate at e.g. acidic pH such as the pH within an endosome, releasing the payload of mRNA. Issued United States patents U.S. Pat. Nos. 7,510,731, 7,718,193, 9,895,451 and 10,709,791 describe examples of temperature and pH sensitive micelles. In particular, issued US patent U.S. Pat. No. 8,747,904 describes polymeric micelles designed for polynucleotide encapsulation. The complete contents of each of these issued US patents is hereby incorporated by reference in entirety. In particular embodiments.

Polymeric micelles are generally formed by the self-assembly of specific copolymers and the supramolecular structure of the micelles has the advantage of specific targeting, as micelle dissociation or change in conformation can be triggered by varying environmental conditions such as temperature and pH. pH sensitive polymeric micelles are synthesized using either acid-sensitive precursors or base sensitive precursors for them to be sensitive to a specific pH or pH range of interest.

Liposomes and micelles are both nanoparticles that are capable of drug delivery and they can be used together in the present methods. They both have an amphiphilic structure and nano-size diameters that can be used in biological applications. Liposomes have a lipid bilayer that separates an aqueous internal compartment from the bulk aqueous phase, while micelles have lipid monolayer with a hydrophobic core and hydrophilic surface. Also, liposomes can deliver a variety of molecules while micelles are often used to deliver water insoluble molecules. The most common micelles are spherical but others can be used as well. If used together in the practice of the present methods, the LNPs can accommodate a hydrophilic payload (e.g. mRNA with or without hydrophilic modifications) while the micelles may contain mRNA that is modified to be less or non-water soluble.

Dendrimers: Have treelike structure and hollow core. Dendrimers are highly branched synthetic polymers of nanometer dimension. They are composed of multiple highly branched monomers that emerge radially from the core. Their well-defined composition, monodisperse size, modifiable surface functionality, multivalency, water solubility, and available internal cavity make them suitable for drug delivery. They are similar to micelle nanoparticles as they can have a hydrophobic core and a hydrophilic surface, and they can be used to prepare stable polymeric micelles like nanoparticles, which are effective for delivering drugs such as the mRNA described herein, especially those that are poorly soluble in water (e.g. mRNA modified to be less hydrophilic/more hydrophobic). depending on their composition, the three macromolecular architectural classes generate polydisperse products of different molecular weights and varying biological properties such as polyvalency, self-assembling, electrostatic interactions, chemical stability, low cytotoxicity, and solubility. These varied characteristics make dendrimers a good choice for drug delivery. See, for example, issued United States patents U.S. Pat. Nos. 9,345,781, 10,561,673, 11,446,238 and 11,918,657, the complete contents of each of which is hereby incorporated by reference in entirety.

Cyclodextrins: Cyclodextrins (CDs) are cyclic oligosaccharides that are used in drug delivery to improve the stability, solubility, and bioavailability of drugs. CDs may be used to deliver the mRNA disclosed herein, cither alone or in combination with another type of nanoparticle or as a component of another type of nanoparticle.

Polymersomes or polymeric vesicles: Polymer vesicles are nanoparticles with hollow nanostructures having a hydrophilic cavity and hydrophobic membrane somewhat similar to reverse micelles. Polymersomes are used in drug delivery systems due to their good biocompatibility, stability, and versatility, depending on the composition and molecular weights of the copolymers. Polymersomes are excellent drug delivery vehicles since they are “tunable” (due to the versatility of monomers and the possibility to change block polymers' molar mass and percentage), have a low critical aggregation concentration, and have a robust bilayer. The polymersomes' hollow core can be used to encapsulate hydrophilic compounds, and the bilayer can be dedicated for loading the hydrophobic compounds. See, for example, issued United States patent U.S. Pat. No. 10,987,409 and published US patent applications US20230241219 and US20220273792, the complete contents of each of which is hereby incorporated by reference in entirety.

Other suitable carriers of the mRNA include but are not limited to exosomes, polymersomes and poly(beta-amino ester) s (PBAE) s, as described in the definition's section herein.

### Formulation

The LNPs and/or micelles, liposomes, etc. are typically delivered or administered in a pharmaceutical composition. Such pharmaceutical compositions generally comprise at least one or more of the disclosed and substantially purified mRNAs in an LNP carrier. Accordingly, the present disclosure encompasses such formulations/compositions. The compositions generally include a pharmacologically suitable (physiologically compatible) carrier. In some aspects, such compositions are prepared as liquid solutions or suspensions. Solid forms suitable for solution in, or suspension in, liquids prior to administration are also contemplated (e.g., lyophilized forms of the nucleic acids), as are emulsified preparations. In some aspects, the formulations are liquid and are aqueous or oil-based suspensions or solutions.

In some aspects, the active ingredients are mixed with excipients which are pharmaceutically acceptable and compatible with the active ingredients, e.g., pharmaceutically acceptable salts. Suitable excipients include, for example, water, saline, dextrose, glycerol, ethanol and the like, or combinations thereof. In addition, the composition may contain minor amounts of auxiliary substances such as wetting or emulsifying agents, pH buffering agents, preservatives, and the like. If it is desired to administer an oral form of the composition, various thickeners, flavorings, diluents, emulsifiers, dispersing aids or binders and the like are added. The composition of the present invention may contain any such additional ingredients so as to provide the composition in a form suitable for administration. The final amount of compound in the formulations varies but is generally from about 1-99%. Still other suitable formulations for use in the present invention are found, for example in Remington's Pharmaceutical Sciences, 22nd ed. (2012; eds. Allen, Adejarem Desselle and Felton).

Some examples of materials which can serve as pharmaceutically acceptable carriers include, but are not limited to: ion exchangers, alumina, aluminum stearate, lecithin, serum proteins (such as human serum albumin), buffer substances (such as Tween® 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

“Pharmaceutically acceptable salts” of the compounds refers to the relatively non-toxic, inorganic and organic acid addition salts and base addition salts of compounds of the present disclosure. In some aspects, these salts are prepared in situ during the final isolation and purification of the compounds. In particular, acid addition salts can be prepared by separately reacting the purified compound in its free base form with a suitable organic or inorganic acid and isolating the salt thus formed. Exemplary acid addition salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, sulfamates, malonates, salicylates, propionates, methylene-bis-β-hydroxynaphthoates, gentisates, iscthionates, di-p-toluoyltartrates, methanesulfonates, ethanesulfonates, benzenesulfonates, p-toluenesulfonates, cyclohexylsulfamates and laurylsulfonate salts, and the like. See, for example S. M. Berge, et al., “Pharmaceutical Salts,” J. Pharm. Sci., 66, 1-19 (1977) which is incorporated herein by reference. Base addition salts can also be prepared by separately reacting the purified compound in its acid form with a suitable organic or inorganic base and isolating the salt thus formed. Base addition salts include pharmaceutically acceptable metal and amine salts. Suitable metal salts include sodium, potassium, calcium, barium, zinc, magnesium, and aluminum salts. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which have sufficient basicity to form a stable salt, and preferably include those amines which are frequently used in medicinal chemistry because of their low toxicity and acceptability for medical use. ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabictylamine, N-ethylpiperidine, benzylamine, tetramethylammonium, tetraethylammonium, methylamine, dimethylamine, trimethylamine, ethylamine, basic amino acids, e.g., lysine and arginine, and dicyclohexylamine, and the like. In some aspects, the salt is an HCl (hydrochloride) salt.

The compounds may also be formulated for delayed, long-acting and/or sustained release. Those of skill in the art are aware of such formulations, including those described in published United States patent application US20220304938A1 and issued U.S. Pat. No. 10,322,089B2, the complete contents of each of which is hereby incorporated by reference in entirety.

The lipid delivery systems disclosed herein may be altered by techniques known to those of skill in the art to improve or optimize any of several parameters including but not limited to: to increase circulation time, to improve delivery efficacy, to increase or decrease mRNA loading, to improve storage times, to render the coast more economical, etc. This is typically accomplished by varying the type and/or chain length of the lipids, by using combinations of different lipids such as phospholipids and simpler lipids, etc.

### Administration

The pharmaceutical compositions disclosed herein are administered in vivo by any suitable route including but not limited to: inoculation or injection (e.g. intravenous, intraperitoneal, intramuscular, subcutaneous, and the like), and/or by absorption through epithelial or mucocutaneous linings (e.g., nasal, oral, gastrointestinal mucosa, and the like). Other suitable means include but are not limited to: inhalation (e.g. as a mist or spray), orally (e.g. as a pill, capsule, liquid, etc.), intranasally, etc. In preferred embodiments, the mode of administration is by nasal inhalation, pulmonary delivery, intraperitoneal delivery, or sub-cutaneous delivery.

In addition, the compositions may be administered in conjunction with other treatment modalities such as agents that target: seizures, behavioral symptoms (ADHD, OCD, anxiety), sleep and GI disorders, dietary supplements, etc. Examples include but are not limited to vigabatrin, valproic acid, various targeted therapeutics e.g. NCS-382, bumetanide, ganaxolone, farnesol, taurine, torin-2, SGS-742, enzyme replacement therapy (ERT; although this may not be necessary when the mRNA disclosed herein is administered), agents that trap aldehydes, one or more mTOR inhibitors and/or GABA-T inhibitors. Suitable mTOR inhibitors include rapamycin, while suitable GABA-T inhibitors include vigabatrin. If administered by infusion, drugs that reduce the risk of infusion-related reactions, such as dexamethasone, acetaminophen, diphenhydramine or cetirizine, and ranitidine, may also be administered.

A “therapeutic” or “therapeutically effective dose” or an “effective amount” of mRNA is a dose sufficient to elicit or cause a desired result, such as an abatement, decrease, elimination, etc. of at least one symptom of the disease that is being treated, i.e., SSADHD. The amount is sufficient to cause an observable change, be it a macroscopic or a microscopic change. Preferably, a phenotypic change is observed in response to the treatment. The term “therapeutically effective amount” refers to that amount of a therapeutic agent, at dosages and for periods of time necessary, that is effective to “treat” (e.g., alleviate or at least lessen symptoms of a disease or disorder in a subject (e.g., a mammal). A “prophylactically effective amount” refers to an amount effective, at dosages and for periods of time necessary, to achieve the desired prophylactic result, i.e. to prevent or lessen the severity of at least one symptom of the disease or condition.

Symptoms of SSADHD that can be treated after their occurrence and/or before their occurrence (e.g., treated in utero, during infancy, or in early childhood such as at an age of about 1, 2, 3, 4, 5, 6, 7, 8, 9, or 10) include but are not limited to: developmental delays, especially in speech development; intellectual disabilities; decreased muscle tone (hypotonia) soon after birth; seizures, difficulty coordinating movements (ataxia), decreased reflexes (hyporeflexia), behavioral problems, sleep disturbances, hyperactivity, difficulty maintaining attention, anxiety, other behavioral and psychiatric features such as aggression and obsessive-compulsive disorder (OCD), etc.

For the treatment of SSADHD, a single dose or mRNA as disclosed herein typically ranges from about 0.05 to about 5.00 mg of mRNA per kg of body weight of the subject (mg/kg), such as about 0.05, 0.075, 0.10, 0.15, 0.20, 0.25, 0.3, 0.35, 0.40, 0.45, 0.50, 1.00, 1.25, 1.50, 1.75, 2.00, 2.25, 2.50, 2.75, 3.00, 3.25. 3.50, 3.75, 4.00. 4.25, 4.50, 4.75 or 5.00 mg/kg. In some aspects, the amount is from about 0.10 to about 2.00 mg/kg.

Those of skill in the art will recognize that the clinical presentation of SSADHD is variable and varies from very severe to mild, depending on the exact mutation that has occurred. (is mutation-dependent). Doses are typically higher than 2.0 mg/kg if severe disease is present (e.g. up to about 5.00 mg/kg). Further, dosing is adapted to the age at which mRNA therapy is initiated: individuals with SSADHD have high GABA and GHB tissue and blood concentrations at birth but those levels decrease with age, although they never normalize. Thus, mRNA therapy is adjusted for the maturational age of the patient in that doses are typically higher in younger patients than in older ones. For example, in some aspects, from about 1-10 mg/kg mRNA (e.g. about 1, 2, 3, 4, 5, 6, 7, 8, 9, or 10 mg/kg) is administered as soon as the diagnosis is made, typically and on average between 1 and 3 years of age, and this amount is decreased to from about 0.05 to about 0.5 mg/kg after puberty, such as to about 0.05, 0.10, 0.15, 0.20, 0.25, 0.30, 0.35, 0.40, 0.45 or 0.50 mg/kg. In some aspects, about 5 mg/kg mRNA is administered as soon as the diagnosis is made, typically and on average between 1 and 3 years of age, and that amount is tapered down to about 0.1 mg/kg after puberty. Multiple administrations may be required. This is best determined by a health professional such as a physician who administers the mRNA observes the results. Administration may be required, for example, weekly, monthly, every other month, yearly, etc. After puberty is complete, it may be possible to cease administration.

In some aspects, complete and/or partial rescue of the phenotype of the disease depends on mRNA intervention early enough during development to prevent irreversible programing of GABA-dependent pathways. Thus, in some aspects, the treatment is provided before birth, i.e. in utero or even via gene editing before or during in vitro fertilization procedures, e.g. following genotyping of a fetus, an egg, the donor parents, etc. to determine the risk, likelihood or certainty of a genetic mutation that would result in SSADHD.

### Examples

### Example 1

### Materials and Methods

The lipids that were used are as follows: (4-(dimethylamino)-butanoic acid, (10Z,13Z)-1-(9Z,12Z)-9,12-octadecadien-1-yl-10,13-nonadecadien-1-yl ester (DLin-MC3-DMA), cholesterol, 1,2-dimyristoyl-rac-glycero-3-methoxypolyethylene glycol-2000 (DMG-PEG2k), and 1,2-distearoyl-sn-glycero-3-phosphocholine (DSPC)).

LNPs studied were composed of DLin-MC3-DMA ((6Z,9Z,28Z,31Z)-heptatriacont-6,9,28,31-tetraene-19-yl4-(dimethylamino) butanoate), cholesterol, DMG-PEG2K (1,2-dimyristoyl-rac-glycero-3-methoxypolyethylene glycol-2000), and DSPC (1,2-distearoyl-sn-glycero-3-phosphocholine). Typical LNP particle sizes range between 80 nm and 100 nm with narrow distribution (PDI<0.05) and a high (>95%) mRNA encapsulation efficiency.

mRNA-containing hALDH5A1-LNPs were prepared using microfluidic mixing as described by Herrera, 2021 (Biomater. Sci., 2021,9, 4289-4300). Briefly, all lipids were dissolved in pure ethanol at 50:38.5:1.5:10 molar ratio (DLin-MC3-DMA: cholesterol: DMG-PEG2k: DSPC), and mRNA was diluted in sterile 50 mM citrate buffer. The lipid and mRNA solutions were then mixed using the NanoAssemblr® Benchtop at a 1 (lipids): 3 (mRNA) ratio, followed by a 10-fold dilution with sterile PBS. The resulting LNP solutions were concentrated using Amicon® Ultra Centrifugal filter units (10,000 Da MW cut-off, Millipore). LNP size distribution and zeta potential were determined via dynamic light scattering using a Zetasizer Nano ZSP (Malvern Instruments, UK) and mRNA encapsulation efficiency was determined using Quant-It™ Ribogreen RNA (Invitrogen, Carlsbad, CA). Typical particle sizes ranged between 80 and 100 nm with a narrow distribution (PDI<0.05) and a high (>95%) mRNA encapsulation efficiency.

mRNA transfection of human patient fibroblasts (BCH and HNDS) and aldh5al-KO mouse (lung) fibroblasts was performed using a Lipofectamine™ kit.

In vivo studies were conducted in the well-characterized animal model of SSADH deficiency, the aldh5a1^{-/-} mouse. The phenotype of this strain is remarkably similar to that of severe human SSADHD. The mice rarely live beyond weaning DOL22 after the onset of ataxia and seizures around DOL16 and they show significant stunted preweaning growth, rendering experiments challenging.

Statistical analyses were used with customary levels of β=0.80 (likelihood) and α=0.05 (significance). Animal subjects were randomly assigned to the various study groups using the GraphPad random assignment tool. All experimental data points were included in statistical analyses unless disqualified as outliers using an unbiased outlier test. Equal number of males and females were represented, as much as mating randomness allows. All reagents were rigorously validated prior to usage. Relevant biological variables (covariates) include Sex and Litter.

### Results

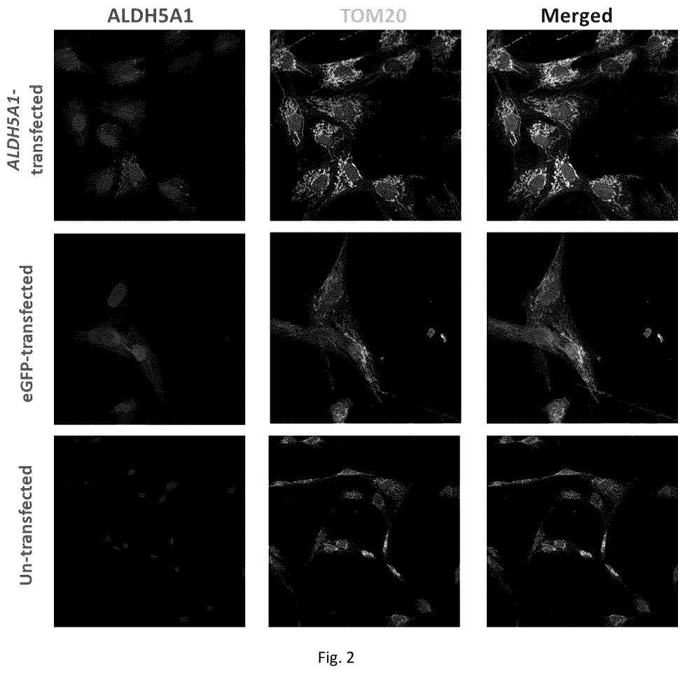

An investigation of the therapeutic potential of ALDH5A1-mRNA treatment in SSADHD in the aldh5a1^{-/-} mouse model and in skin fibroblasts obtained from patients with a confirmed SSADHD diagnosis was undertaken. The work showed that cells (4 different patient-derived fibroblast cell lines and KO mouse lung fibroblasts) transfected with codon-optimized (TriLink®) mRNA coding for human ALDH5A1 (hALDH5A1) expressed ALDH5A1 with mitochondrial subcellular localization as demonstrated by colocalization with the mitochondrial marker, TOM20 (FIG. 2). Measurement of ALDH5A1 enzymatic activity (NADH fluorometric assay) in cell homogenates and of GABA and GHB in cell conditioned media further showed significant expression of enzymatically active ALDH5A1 (for NADH, FIG. 3(A)), and improvement (decrease) in GABA and GHB accumulation (FIGS. 3(B) and (C)).

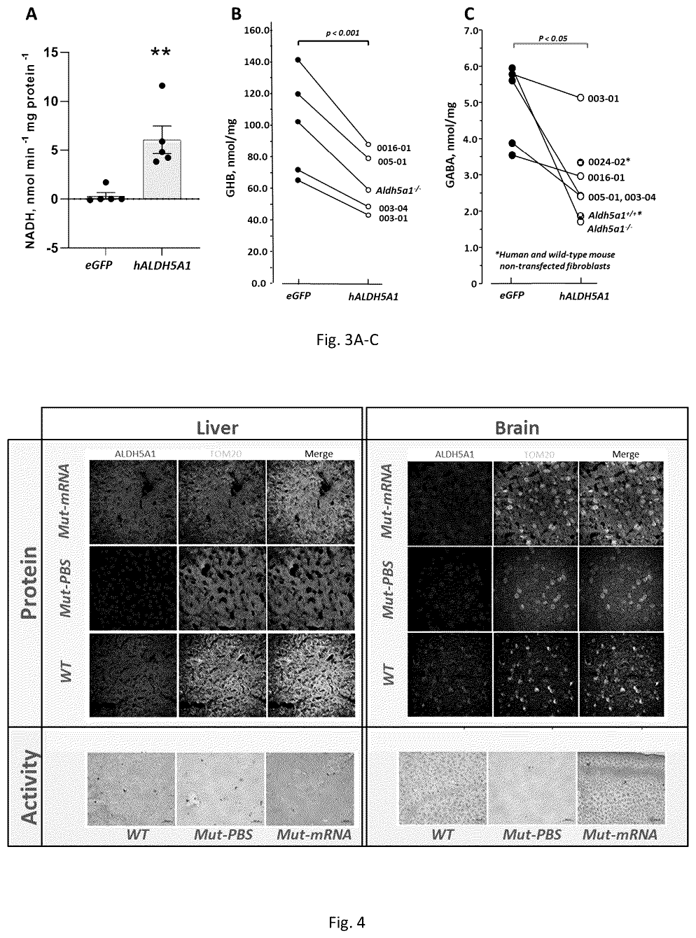

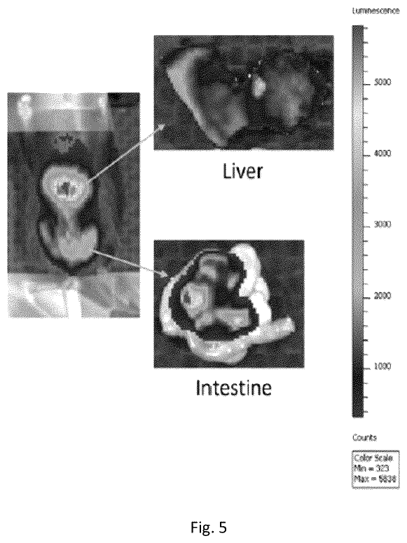

To demonstrate the mRNA approach in vivo, mRNA delivery using lipid nanoparticles (LNPs) was investigated. Two delivery routes were tested: intravenous (i.v., retroorbital) (FIG. 4) using hALDH5A1-LNPs and intraperitoneal (i.p.) using Fluc-LNPs (firefly luciferase mRNA, TriLink®) (FIG. 5). The LNPs used for the delivery of both mRNAs (h ALDH5A1 and Fluc) were identical and administered to aldh5a1^{-/-} mice. The results confirmed successful tissue delivery of both mRNAs to aldh5a1^{-/-} mice (FIGS. 4 and 5).

FIG. 4 shows successful delivery of hALDH5A1 mRNA in the liver and brain of aldh5a1^{-/-} mice following intravenous injection of hALDH5A1-LNPS. Strong expression of ALDH5A1 was documented 48 hours after injection by immunohistochemistry (IHC) and enzyme histochemistry (EHC) in both the liver (FIG. 4, left panel) and the brain (FIG. 4, right panel). Expression in the brain was welcome but surprising as the crossing of the blood-brain barrier (BBB) by the LNPs had not been reported. The discrete expression of enzymatically active ALDH5A1 in the brain after treatment is also notable. It is well known by those skilled in the art that BBB permeability is increased by inflammation of the brain such as after ischemic stroke. There is evidence that brain inflammation (astrogliosis) occurs in human and experimental (mouse) SSADH deficiency (Brown M N, Gibson K M, Schmidt M A, Walters D C, Arning E, Bottiglieri T, Roullet J-B. Cellular and molecular outcomes of glutamine supplementation in the brain of SSADH-deficient mice. JIMD Reports Jun. 15, 2020, Walters D C, Lawrence R, Kirby T, Ahrendsen J T, Anderson M P, Roullet J-B, Murphy E J, Gibson K M. Post-mortem analyses in a patient with succinic semialdehyde dehydrogenase deficiency (SSADHD). II Histological, lipid, and gene expression outcomes in regional brain tissue. J. Child Neurol., 2021). It follows that hALDH5A1-LNPs will be particularly efficient in treating brain-related symptoms in severe SSADH deficiency. It also follows that the potential for side-effects caused by overexpression of active ALDH5A1 in the brain following hALDH5A1-LNP administration at treatment onset will be reduced as brain-related symptoms and inflammation are successfully treated and BBB permeability is restored (decreased) to normal.

FIG. 5 shows successful expression of enzymatically active firefly luciferase in the gut and liver of aldh5a1^{-/-} mice 24 hours after i.p. injection of Fluc-LNPs. Fluc-mRNA was delivered using lipid nanoparticles of the same composition as those used to deliver h ALDH5A1 mRNA through the intravenous route. This demonstrates that intraperitoneal injections of hALDH5A1 mRNA-LNPs will result in the expression of functional human ALDH5A1 in both liver and intestine.

Table 1, below, shows that ALDH5A1 mRNA transfection significantly increases ALDH5A1 activity in human patient fibroblasts (BCH and HNDS) and aldh5a1-KO mouse (lung) fibroblasts. eGFP mRNA transfected cells served as the control group.

TABLE 1

| Patient/Mouse | ALDH5A1* | eGFP* |
|---|---|---|
| Patient 1 | 4.2 | 0.0 |
| Patient 2 | 11.6 | 0.0 |
| Patient 3 | 5.9 | 1.7 |
| Patient 4 | 4.8 | −0.1 |
| Aldh5a1^{-/-} mouse | 3.8 | 0.0 |

*Enzyme activity in nmol min⁻¹ mg protein⁻¹
Data are presented as mean ± SEM, n = 3 independent experiments, measured in triplicate.
**p < 0.01 compared to corresponding control group of eGFP mRNA transfection, p values obtained from unpaired two-sided t test with a 95% confidence interval.

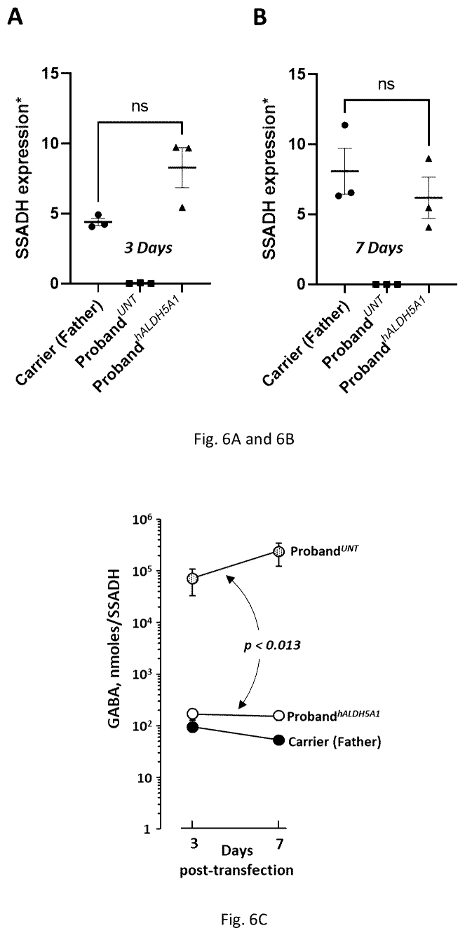

FIG. 6A-C shows successful and prolonged in vitro SSADH protein expression in iPSC-derived SSADHD neurons transfected with human ALDH5A1 mRNA. Two iPSC-derived neuronal cell lines were created from the father carrier and one from the child with SSADHD and were studied with and without transfection with hALDH5A1 mRNA As can be seen in (A), transfection induced SSADH (ALDH5A1) protein expression in transfected neurons and (B) normalized GABA levels in culture media, consistent with significant expression of immunoreactive SSAHD. This finding is consistent with successful phenotypic rescue that lasts at least 7 days in vitro. hALDH5A1-LNPs thus can be used to reestablish neuronal health in SSADHD.

In summary, these show that administration of hALDH5A1-LNPs results in hepatic and brain expression of active ALDH5A1 in an art-recognized animal model of SSADHD. Expression leads to significant decreases in tissue GABA and GHB and improvement of the neurometabolic phenotype of the disease.

### Example 2

Synthesis of pH sensitive micelle: Micelles molecules developed are pH sensitive and depend upon the co-polymers selected for micelle synthesis various routes such as Reversible Addition Fragmentation Chain Transfer (RAFT), nitroxide-mediated polymerization (NMP), atom transfer radical polymerization (ATRP) ring opening polymerization. Polymer Conjugation can be adopted individually or in combinations. pH responsive micelles commonly have ionizable acidic or basic residues whose ionization depends on the pH of solution. We have synthesized the pH sensitive PLA-PEG based micelles.

### Step 1 Polymer Conjugation:

Di-block copolymers are prepared by ring opening polymerization (ROP) method using dl-lactide monomers, m-PEG and stannous octoate as catalyst, so that dl-lactide monomers were grown from one end of m-PEG. Their structure was evaluated using NMR and FTIR. Once we confirmed the conjugation, the polymer conjugate was purified using dialysis technology in DMSO/water using standrad laboratory protocol. Once purified conjugate is obtained it can be then dispersed in water for self assembly.

Step 2 micellization: Polymer conjugates are dispersed at critical micelle concentration in water using sonication or magnetic stirring or a combination of two techniques. We dispersed the PLA-PEG polymer conjugate synthesized in step one in DI water @˜2weight % and sonicated for 20 minutes at room temperature followed by magnetic stirring for 2 hours. As these are self-assembled molecules, we confirmed the size using dynamic light scattering (DLS). The average range for the hydrodynamic diameter was from about 350-400 nm and when very large molecules were observed (e.g. >about 400 nm) we repeated the sonication step and stirring. Once the requisite size micelles were produced, they were frozen at −80° C. and lyophilized to obtain a fluffy powder that was stored in a regular refrigerator at a temperature of 0° C. or below for future use. These powders immediately disperse in water to form micelles. The shape/morphology was determined using microscopy that shows spherical shaped nanoparticles. It is noted that other morphological shapes can be adopted for self-assembled particles.

Step 3 pH sensitivity: Acid- or base-sensitive micelles can be made. Acid-sensitive micelles are preferable for delivery to tissue or intracellular environments that are acidic in nature, Such micelles maintain a size ranging from 150 and 200 nm in physiological solutions at neutral pH. When pH decreases, the micelles first expand in size, change their conformation then open up at pH 3.5, allowing the gradual release of their payload. Base-sensitive micelles are stable in acidic pH, increase their size when pH increases, and deliver their payload (e.g. ALDH5A/mRNA) when the pH rises above neutral pH. Such base-sensitive micelles are stable in acid pH e.g. the gastric milieu and are suitable for delivery of their cargo to the intestine.

### Example 3

Further in vivo testing is conducted to reveal the durability of ALDH5A1 expression after a single injection of ALDH5A1-LNPs and variability depending on the dosage and the subject's age at which the injection is performed. A comparison of intraperitoneal (i.p.) injections of ALDH5A1-LNPs to intravenous injections yields significant metabolic “rescue” of the disease and provides a less invasive yet comparable delivery alternative. Basic toxicity experiments are conducted and show that the LNPs are not toxic. Data on clinical (growth and survival) and histological (brain astrogliosis) are obtained from a multiple injection experimental paradigm.

Pharmacokinetic (PK) studies include studies after i.v. injections, using three (n=3) aldh5a1^{-/-} mice per time-point (3 time-points: 12, 24 and 48 hrs), per dose (4 doses: 0 (PBS, vehicle), 0.1, 1.0 and 2 mg mRNA/kg BW), and per age at injection (Day of Life (DOL) 10, DOL15, and DOL20) (total of 10 experimental groups, 3 variables ([time], [dose], [age]) and 108 mice). The chosen RNA concentrations are in keeping with the range reported by others in rodent models. Multifactor ANOVA statistics with significance level set at p≤0.05 determine the effects of [time], [dose], [age], and interactions between these variables. Areas under the curve (AUCs) and hALDH5A1 maximum gene expression are calculated with a focus on liver and whole brain tissue. For PK studies after i.p. injections, the design also has 3 mice/group, but only one dose is tested, that which gives the greatest AUC for h ALDH5A1 gene expression in the liver after i.v. injection in animals of matching age (total of 6 experimental groups, 2 variables ([time], [age]) and 60 mice).

Survival and growth studies. Two experimental groups of 15 (n=15/group) aldh5a1^{-/-} mice, are tested one receiving hALDH5A1-LNPs and the other receiving PBS (vehicle). Only one mRNA dose (1 mg/kg BW) is tested. Intraperitoneal injections are performed at DOL5, 10, 15, 20 and every 5 days until DOL30 on any surviving mice. Group assignment is random and performed when pregnancy is noted. Injections at DOL5 are performed in all pups of the litter whether they are wild-type, heterozygous or mutants. Genotyping takes place at DOL10. From then on, only aldh5a1^{-/-} mice receive treatment (mRNA or PBS). Body weight is measured at time of injection to adjust dosing. The proportion of surviving mice at DOL 30 in the mRNA treated group is compared with that of surviving mice in the PBS-treated group. In the unlikely event that any PBS-treated mice survive until DOL30, the research team looks for a 50% survival rate at DOL30 in the mRNA-treated group to confirm significant survival benefit (χ² at p≤0.05 (only ˜25% survival rate if there are no survivors in the control group). Kaplan-Meier analysis determines the number of days at which 50% of the mice in each group are still alive with survival curves being compared using the Log Rank test. Surviving mice are euthanized and blood and tissues collected at DOL30.

Another mouse model, the aldh5a1^{STOP/STOP} mouse, was recently developed and is used for SSADHD investigations. Further and notably, the aldh5a1^{/STOP/STOP} mouse also does not survive beyond DOL21-22

With the challenge of i.v. injections in KO mice younger that DOL10, ethical considerations steer a choice to not allow multiple r.o.-i.v, procedures on the same mouse (the procedure requires anesthesia, and the KO mice are very sensitive to stress) and to not genotype pups before DOL10. With a designed time-course of 48 hours for the PK studies, DOL10 is chosen as the earliest age at which the mice can be treated with a single i.v. injection of hALDH5A1-LNPs, and DOL20 the latest age at which to test the LNPs either i.v. or i.p., so that euthanasia and sample collection of the 48-hr time point would not fall beyond their typical “natural” death. Intraperitoneal injections do not require anesthesia and are performed at an earlier age and multiple times on the same animal. Hence the i.p. route is deemed to be the best method to use to gain insight into the long-term benefits of mRNA therapy in developing/maturing (younger) patients with SSADHD. Further, i.p. injections deliver ALDH5A1 mRNA to the gut. Restoring ALDH5A1 activity in the gut is beneficial especially in helping KO mice survive beyond weaning. Notably, in one report on mRNA therapy in a murine model of galactosemia, a single i.p. injection of hGALT-LNPs reversed neonatal galactose sensitivity, promoted growth and increased survival beyond weaning. The mechanism of this phenotypic rescue of galactosemic mice is not clear. However, recent studies suggest that exposure to the intestinal flora that develops during weaning triggers a strong immune response and later-life programming of the immune system. Other studies have shown that normal GABA signaling through GABA receptors is necessary for a normal immune response to microbes. These studies raise the possibility that the immune reaction that occurs at weaning is abnormal in aldh5a1^{-/-} mice (their GABA receptors are down-regulated), leaving them unable to cope with the new intestinal microbiota and causing early death. The microbiota hypothesis is the rationale for selecting i.p. injections and measurement of ALDH5A1 expression in the gut. Confirmation of ALDH5A1 expression in the gut after i.p. injection of hALDH5A1-LNPs is ongoing. However, i.p. injection of Fluc-LNPs results in the expression of active luciferase in both the liver and the gut. Therefore i.p. delivery of hALDH5A1 mRNA using the same LNPs will also result in successful expression of active ALDH5A1 in these tissues. Prenatal mRNA delivery is also contemplated.

### Example 4

End-points are selected to guide the choice of efficacy/toxicity markers of hALDH5A1-mRNA LNP therapy for clinical trials. Study endpoints are summarized in Table 2 (1, liver, br, brain; bl, blood; g, gut):

TABLE 2

| End-points | Method | Specimen |
|---|---|---|
| Disease biomarker: | ELISA | l, br, g, bl |
| GABA, GHB, GABA- | [¹H]NMR | Urine |
| derived metabolites | | |
| Disease biomarker: | ELISA | l, br, bl |
| Glutamine, glutamate | | |
| GABA-related genes | RT-qPCR | l, br, g |
| (gabra1-6; glul; gad1; | | |
| gls) | | |
| ALDH5A1 expression | PCR; ELISA*; | l, br, g |
| (mRNA, protein, activity) | NADH**; | |
| | MTT*** | |
| Astrogliosis | IHC (GFAP | br |
| | labeling) | bl |
| | ELISA (GFAP) | |
| Survival | Kaplan-Meier | Mouse |
| Toxicity (ASAT; ALAT; | Sigma kits; | l, br, g, bl |
| creatinine; cytokines) | ELISA | |

*Human SSADH, mitochondrial (Aldh5A1) ELISA kit;
**tissue homogenates;
***histology

Such markers include blood GABA and GHB, and brain GABA measured by [¹H]-MRS, metabolites shown to correlate with neurophysiological outcomes in patients enrolled in the ongoing Natural History study of SSADHD. Brain astrogliosis is challenging to assess in humans yet recent studies have reported the use of PET imaging to quantify the glial response to inflammatory stimuli such as those triggered by Aβ plaques in Alzheimer's disease. PET imaging has been used to assess GABAA receptor expression in patients with SSADHD, and thus is also used to monitor the efficacy of mRNA therapy on brain astrogliosis in clinical trials. Non-invasive although indirect imaging methods such as [¹H]-MRS or resting state fMRI also provides information on impaired glia-neuron functional connectivity. The focus is on brain glutamine and astrogliosis since hALDH5A1-LNPs predominantly targets the liver and compelling preliminary data shows strong expression of ALDH5A1 in the brain after LNP delivery. If expression of active enzyme in the brain is modest compared to that in the liver, it can still improve reactive astrogliosis and CNS dysfunction.

Toxicity of mRNA therapy is addressed in pre-clinical studies during drug development. The repeated dosage (i.p.) studies together with the measurement of inflammatory cytokines and markers of liver and kidney function (transaminases, creatinine) provide useful insight into safety, tolerability, or untoward side-effects. For example, circulating and neutralizing anti-h ALDH5A1 antibodies are also detected. Past work on mRNA therapy suggests that the occurrence of antibodies is not a major concern.

During fetal and early postnatal life, GABA acts as an excitatory neurotransmitter regulating neurogenesis and CNS maturation. It follows that ALDH5A1 deficiency together with GABA and GHB tissue accumulation in embryos likely alters neurogenesis and CNS maturation, leading later in life to the neurobehavioral manifestations so prevalent in SSADHD. In this context, mRNA therapy started too early in life while the CNS is still maturing, may provide metabolic rescue but could also negatively impact GABA-dependent maturational processes and lead to unpredictable consequences later in life. Such concern is not unique to mRNA therapy but also applies to gene therapy or ERT.

The mRNA trial complements an on-going natural history study which developed a clinical severity score (Tokatly Latzer I, Roullet J-B, Gibson K M, Pearl P L. Establishment, and validation of a clinical severity scoring system for succinic semialdehyde dehydrogenase deficiency. J. Inherit. Metab. Dis., 2023 September; 46 (5): 992-1003) with the intent to use it as an efficacy monitoring tool in future trials and already identified an array of systemic biomarkers also relevant to therapeutic monitoring. The natural history study is also geared toward understanding the SSADHD brain with a comprehensive set of serial neurophysiological and imaging investigations such as but not limited to transcranial magnetic stimulation, high-density EEG, brain GABA-[¹H]MRS, functional MRI and MEG). Such non-invasive methods are used to reveal the benefits or side-effects of mRNA therapy on the CNS of the research group's patients.

Clinical studies are used to optimize administration of mRNA variable dosing at different times of life, building on evidence that: (1) blood GABA and GHB levels are high at birth and decrease with age, and (2) the prevalence of disease symptoms appears to rise when GABA and GHB decrease (around the age of 8-12 years). An mRNA therapy dose titration scheme is adapted for the metabolic changes in GABA and GHB.

### Example 5

A human subject is diagnosed with SSADHD before birth (e.g. via amniocentesis) or while less than 1 year of age. ALDH5A1d mRNA is administered to the subject using one or more polymer-based nanoparticles, or mixtures thereof (e.g. via LNPs, micelles, liposomes, exosomes and or dendrimers). A tapered regime is used for dosing and multiple administrations continue a needed until the subject reaches puberty and thereafter as needed, e.g. beginning with about 5 mg mRNA/kg body weight and ending with about 0.1 mg/kg. As a result of administration, at least one symptom of SSADHD is lessened or does not develop further or does not develop at all in the subject.

In these claims, reference to an element in the singular is not intended to mean “one and only one” unless explicitly stated, but rather “one or more.” All structural, chemical, and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element is to be construed as a “means plus function” element unless the element is expressly recited using the phrase “means for . . . ” No claim element is to be construed as a “step plus function” element unless the element is expressly recited using the phrase “step for . . . .”

Although the description here contains many details, these should not be construed as limiting the scope of the disclosure but as merely providing illustrations of some of the presently preferred embodiments. Therefore the scope of the disclosure encompasses other embodiments which may become obvious to those skilled in the art.

We claim:

1. A method for treating succinic semialdehyde dehydrogenase deficiency (SSADHD) in a subject in need thereof, comprising administering to the subject a therapeutically effective amount of an mRNA coding for aldehyde dehydrogenase 5 family member A1 (ALDH5A1).

2. The method of claim 1, wherein the mRNA is administered by one or more routes selected from the group consisting of intravascular injection, intrathecal injection, nasal inhalation, pulmonary delivery, intraperitoneal delivery, sub-cutaneous delivery, and oral delivery.

3. The method of claim 1, wherein the mRNA is encapsulated in a delivery medium.

4. The method of claim 3, wherein the delivery medium comprises a plurality of micelles.

5. The method of claim 4, wherein the micelles are pH sensitive micelles.

6. The method of claim 5, wherein the plurality of micelles comprises one or more self-assembling amphiphilic pH-sensitive copolymers containing hyaluronic acid, alginic acid, heparin, esters, acrylates, amino esters, carboxymethyl cellulose, carboxymethyl dextran, poly histidine, poly-vinyl pyridine, hydroxyethyl methacrylate, chitosan, tertiary amine starch, imine, and/or hydrazones.

7. The method of claim 3, wherein the delivery medium comprises one or more of a plurality of phospholipids, fatty acids, or other amphiphiles.

8. The method of claim 3, wherein the delivery medium comprises lipid nanoparticles (LNPs).

9. The method of claim 1, wherein a sequence of the mRNA is at least 95% identical to SEQ ID NO: 1.

10. The method of claim 1, wherein the mRNA codes for a c.354G>C variant of ALDH5A1.

11. The method of claim 1, wherein the mRNA is modified to be self-replicating.

12. The method of claim 1, wherein the step of administering delivers the mRNA to the liver of the subject.

13. The method of claim 1, wherein the step of administering delivers the mRNA to the gut of the subject.

14. The method of claim 1, wherein the step of administering delivers the mRNA to the brain of the subject.
